(19) United States
(12) Patent Application Publication
(10) Pub. No.: US 2025/0281365 A1
(43) Pub. Date: Sep. 11, 2025

(54) SOLID COSMETIC CARE COMPOSITION COMPRISING AT LEAST ONE CAVITY

(71) Applicant: CAPSUM, MARSEILLE (FR)

(72) Inventors: Sébastien BARDON, VEYRIER DU LAC (FR); Mathieu GOUTAYER, SAINT MALO (FR); Jean-François GONIDEC, AIX-EN-PROVENCE (FR)

(21) Appl. No.: 18/859,866

(22) PCT Filed: Apr. 26, 2023
(86) PCT No.: PCT/EP2023/060982

§ 371 Date: Oct. 24, 2024

(30) Foreign Application Priority Data
Apr. 26, 2022 (FR) FR2203882

(51) Int. Cl. A61K 8/02; A61Q 19/10

(52) U.S. Cl. CPC A61K 8/0216; A61Q 19/10; A61K 2800/805; A61K 2800/87

(57) ABSTRACT

The present disclosure relates to a solid cosmetic care composition of a keratin material, in particular the skin and/or the lips, comprising at least one biological/cosmetic active agent and at least one cavity, such that the composition has a porosity of greater than or equal to 20% by volume relative to the total volume of the solid composition, as well as to associated production methods.

The invention relates to a solid cosmetic composition intended for the care of a keratin material, more particularly of the skin and/or of the lips.

Solid cosmetic compositions intended to the care of a keratin material in the form of a stick or powder to be dispersed, generally in water, are already known.

However, such solid care compositions, generally anhydrous, do not give total satisfaction in terms of homogeneity of deposition, of comfort, of sensoriality and/or of hydration. Indeed, the depositions are generally greasy and/or sticky.

Moreover, with regard to sticks, the use thereof involves repeated contact between the solid composition and the treated keratin material which, for obvious reasons, is not very hygienic. What is more, the degradation of the sticks and/or the rupture thereof and/or disintegration before the total consumption there generates wastage and hence waste.

With regard to the powders to be dispersed, same are generally water-soluble or water-dispersible powders, to which it is often difficult to add the right amount of water in order to achieve the desired final liquid composition. Moreover, the dispersion thereof in aqueous medium is often perceived by the user as a deceptive, since not very qualitative, experience. Finally, such procedures make difficult the presence of a fatty (or oily) phase and hence the presence of lipophilic active ingredients. Thereof explains, at least in part, why such type of composition is generally endowed with unsatisfactory sensoriality and effectiveness.

Solid cosmetic care compositions in the form of single doses are already known. However, such compositions comprise a lot of packaging, at least partly disposable and/or have properties that are not entirely satisfactory in terms of gripping, ease of application, sensoriality, hydration and/or good feeling on application.

The present invention aims to overcome the aforementioned drawbacks by proposing solid cosmetic care compositions, more particularly in the form of a single dose, guaranteeing good grip by the user, direct and comfortable application, economical packaging, and which permits the presence of a fatty (or oily) phase and hence the presence of lipophilic active agents.

### COMPOSITION

The invention thereby relates to a solid care cosmetic composition of a keratin material, more particularly for the skin and/or the lips, comprising at least one cosmetic/biological active agent, characterized in that the said composition comprises at least one cavity such that said composition comprises a porosity greater than 60 or equal to 20%, preferably greater than or equal to 30%, better greater than or equal to 40%, more particularly greater than or equal to 50%, more particularly greater than or equal to 70%, and most particularly greater than or equal to 80%, by volume relative to the total volume of said solid composition.

Thereby, a composition according to the invention may remain endowed with a size and/or a volume similar to current solid care compositions but differs in the presence of at least one cavity such that the porosity of said composition is greater than or equal to 20% by volume relative to the total volume of said composition.

“Solid”, as defined by the present invention, refers to a composition which, at ambient temperature and at atmospheric pressure, is not apt to collapse under its own weight. Advantageously, one intends to refer to any composition having a compressive strength of greater than or equal to 20 g, preferably greater than or equal to 50 g, and better greater than or equal to 100 g, at ambient temperature (20-25° C.), after penetration by a cylindrical probe of revolution having a diameter of 0.8 cm into the matrix of the composition in a thickness of 1 mm at a speed of 0.5 mm/s, and removal of said probe from the matrix of the composition at a speed of 0.5 mm/s; the compression strength being measured with an analyzer such as a “LFRA Texture Analyzer” marketed by STEVENS/MECHTRIC.

A composition according to the invention is not in the form of a paste. A composition according to the invention is not a pulverulent composition, and hence is not in the form of a powder. Finally, a composition according to the invention is neither a porous solid derived from the agglomeration of powders. On the other hand, a composition according to the invention may be a puffed composition, and hence may be in the form of a foam. According to a particular embodiment, a composition according to the invention is not a puffed composition, and hence is not in the form of a foam.

Preferably, a solid composition according to the invention is not coated with a water-soluble coating.

“Porosity”, as defined by the present invention, refers to all the voids of a solid material, the voids being able to be filled with fluids, in particular water, when the solid composition is brought into contact with an aqueous solution, e.g. water such as tap water.

“Cavity”, as defined by the present invention, refers to an empty space inside a solid body and which communicates directly with the external medium, like e.g. the cavities illustrated in FIGS. 1 to 3 and 6. A cavity can thus be referred to, without distinction, by the terms “orifice”, “pore” or “hole”. Advantageously, the cavity(ies) is/are through, i.e. the cavity designates an empty space inside a solid body and which communicates directly with the external medium on both sides of the solid body.

Thereby, the volume of the cavity(ies) in a solid composition according to the invention represents at least 20%, more particularly at least 30%, preferably at least 40%, more particularly at least 50%, even at least 60%, more particularly at least 70%, and most particularly at least 80%, of the total volume of said solid composition.

Advantageously, a solid composition according to the invention has a density comprised between 0.2 and 0.8, preferably between 20 and 0.7, more particularly between 20 and 0.6.

A solid composition according to the invention may have a total volume comprised between 0.125 cm³ and 100 cm³, preferably between 0.250 cm³ and 75 cm³, more particularly between 0.5 cm³ and 50 cm³, better between 1 cm³ and 25 cm³, and most particularly between 2.5 cm³ and 15 cm³.

A solid composition according to the invention may have a weight comprised between 0.025 g and 200 g, preferably between 0.25 g and 150 g, more particularly between 0.5 g and 100 g, better between 1 g and 75 g, most particularly between 2.5 g and 50 g, even between 5 g and 25 g (“g” corresponding to the “gram” unit).

Advantageously, a solid composition according to the invention may have a weight comprised between 0.25 g and 200 g, preferably between 0.50 g and 150 g, more particularly between 1 g and 100 g, better between 2.5 g and 75 g, and most particularly between 5 g and 50 g (“g” corresponding to the “gram” unit).

According to a first preferred embodiment, a solid composition according to the invention comprises a ratio between the volume of the cavities and the total volume of the composition comprised between 0.05 and 0.9, preferably between 0.1 and 0.8, more particularly between 0.2 and 0.9, preferably between 0.2 and 0.8, better between 0.2 and 0.7, even between 0.2 and 0.6.

“Total volume” refers to the volume resulting from the sum of the volume of solid and the of the volume of the cavities.

Within the framework of the present invention, the cavities may be considered to be macroporosity, i.e. pores the minimum width, the diameter even, of which is greater than or equal to 0.5 mm, preferably greater than or equal to 0.75 mm, and more particularly greater than or equal to 1 mm, even comprised between 0.5 mm et 2 cm, most particularly between 1 mm et 2 cm, preferably between 2 mm et 1 cm, and more particularly between 3 mm et 75 mm, better between 4 mm et 50 mm, and most particularly between 5 mm et 25 mm. More particularly, the pores have a minimum width, even a diameter, comprised between 0.5 mm et 5 mm, preferably between 1 mm and 3 mm.

As such, the Applicant indicates that, at equivalent porosity, manufacturing a stable solid composition is:
easy in the presence of small cavities, e.g. the cavities present in solid substrates where the porosity is consecutive to the addition of a gas during the manufacturing process, as described in U.S. Pat. No. 8,349,341, but
much more difficult in the presence of macroporosities such as same considered in the present invention.

The presence of cavities in the contents and/or volumes and/or dimensions considered hereinabove makes it possible to obtain solid compositions which require less raw materials while remaining endowed with satisfactory gripping properties. A solid composition according to the invention is thus apt to be put in a single-dose format generating no loss or less loss. Such weight saving further has the advantage of permitting less polluting and less expensive transport. A solid composition according to the invention is thus both ecologically and economically advantageous.

Moreover, the inventors observed an unexpected effect in terms of texture and transformative effect when a solid composition according to the invention is placed in the presence of an aqueous phase. Indeed, the inventors have observed that a solid composition according to the invention, in contact with an aqueous phase, exhibits a faster transformation kinetics between the original solid state and a liquid state, e.g. such as a gel or a cream, compared to a solid composition of the same size and volume, but without any cavity. Without wishing to be bound by any theory, the inventors believe that the presence of cavity(ies) within the solid composition makes it possible to facilitate the transition to a viscous liquid composition, e.g. when the solid composition is mixed with the hands, or leads to an increase in the contact surface area with the aqueous phase, more particularly water, and thus a better hydration of said solid composition.

Thereby, the invention even makes it possible to regulate the speed at which the solid composition will swell and/or melt, by adjusting the developed surface to constant volume, by regulating the percentage of porosity, and more particularly by acting on the shape and/or the number and/or dimensions of the cavities, even the number of walls and/or the thickness of the walls of the cavities.

Advantageously, the size and/or the volume of the cavities can be adjusted to store and/or react with the right amount of water necessary to achieve a final liquid composition endowed with satisfactory/expected properties, even optimized and/or homogeneous (i.e. between different single doses), e.g. in terms of viscosity, hydration, ease of spreading and/or transformative effect.

Thereby, a composition according to the invention even makes it possible to obtain evanescent compositions, namely compositions apt to be transformed into liquid compositions, more particularly in the form of gels or creams, in contact with an adequate quantity of water.

Finally, the inventors have observed that the porous structure of a solid composition according to the invention makes it possible to obtain depositions on keratin materials which are more homogeneous, even without recourse to application members.

Preferably, a composition according to the invention is a single-dose composition. Such embodiment is particularly advantageous from a hygienic point of view in that same prevents repeated contacts between the solid composition and the keratin material. Such embodiment also makes it easy for the user to adapt the desired cosmetic effect according to his/her state of mind (mood), his/her style of clothing and/or the time of day.

Thereby, the invention also relates to a packaging comprising a plurality of solid compositions according to the invention in the form of single doses with different active ingredient/cosmetic effect. Thereby, the invention relates to a packaging comprising a plurality of solid compositions according to the invention, preferably in the form of single doses, said compositions comprising different cosmetic/biological active agents and/or different contents of cosmetic/biological active agent(s). The packaging thus has the advantage of ensuring an easy customization of the care product for the user according to his/her state of mind (mood), his/her style of clothing and/or the time of day.

Preferably, a composition according to the invention is a topical composition. A composition according to the invention is not an oral composition.

A solid composition according to the invention may have any shape, e.g. a spherical, ellipsoid, tetrahedral or polygonal shape, more particularly in the shape of a parallelogram, preferably a rectangular parallelogram.

Preferably, in particular to provide a good grip, a solid composition according to the invention has:
a width, or even a diameter, comprised between 1 cm and 10 cm, preferably between 2 cm and 8 cm, more particularly between 3 cm and 6 cm, and better between 4 cm and 5 cm; and/or
a height (or thickness) of between 0.5 cm and 5 cm, preferably between 1 cm and 4 cm, and more particularly between 2 cm and 3 cm.

The cavity(ies) of a solid composition according to the invention can have any shape, e.g. a round, oblong, ovoid, triangular, polygonal, more particularly hexagonal, honeycomb shape, or in the shape of a paving (or cutout), e.g. of such as a Voronoï diagram, preferably in the shape of a honeycomb, as illustrated in FIGS. 3 and 6.

The cavity(ies) of a solid composition according to the invention can also represent letters or the logo of a brand or a company, as illustrated in FIG. 2.

A solid composition according to the invention comprises one or a plurality of cavities, preferably a plurality of cavities, either identical or different.

According to a first variant, the composition according to the invention comprises at least one cavity, more particularly a through cavity.

According to a second preferred variant, a composition according to the invention comprises a plurality of cavities, more particularly through cavities, as illustrated in FIGS. 3 and 6.

The cavities are through cavities, advantageously along an axis x-x′.

According to a particular embodiment, a composition according to the invention may comprise at least two cavities connected together. Such an embodiment is advantageous because same enhances capillarity phenomena when brought into contact with water, which improves the aforementioned technical effects.

Advantageously, a solid composition according to the invention comprises a plurality of cavities which take a honeycomb-shaped structure, as illustrated in FIG. 3. The honeycomb shape is particularly advantageous in that same permits the manufacture of a solid composition provided with a very advantageous “volume of the cavity(ies)/total volume of the composition” ratio, more particularly greater than or equal to 0.5, even greater than or equal to 0.75, without prejudice to the grip and the robustness of said composition to the mechanical stresses likely to be applied thereto and/or to storage. In other words, the honeycomb structure is an excellent compromise between cavity volume and mechanical strength.

Advantageously, the cavity(ies) of a solid composition according to the invention comprise(s) walls having a thickness comprised between 10 μm and 1,500 μm, preferably between 20 μm and 1,000 μm, more particularly between 30 μm and 750 μm, most particularly between 50 μm and 500 μm, even between 100 μm and 400 μm. More particularly, the cavity(ies) of a solid composition according to the invention comprises/comprise walls having a thickness comprised between 250 μm and 500 μm.

Advantageously, a solid composition according to the invention comprises a plurality of cavities which take a honeycomb-shaped structure comprising walls having a thickness comprised between 10 μm and 600 μm, preferably between 20 μm and 500 μm, more particularly between 30 μm and 400 μm, most particularly between 50 μm and 300 μm, even between 100 μm and 200 μm.

According to a particular embodiment, a solid composition according to the invention, in particular when same takes a honeycomb configuration as described hereinabove, may comprise a plurality of sections, more particularly polygonal, each of the sections defining a breakable perimeter permitting the separation of at least one section from the remainder of the solid composition. Thereby, according to such embodiment, each section of the solid composition corresponds to a single dose.

According to a particular embodiment, a composition according to the invention may also comprise additional patterns distinct from the cavities, said patterns being able, e.g., to be produced by embossing.

According to a first variant, a composition according to the invention is anhydrous. “Anhydrous”, as defined by the present invention, refers to a composition which, before any contact with water, comprises a water content of less than 5% by weight, preferably less than 1% by weight and more particularly less than 0.5% by weight, relative to the total weight of said composition.

According to a second variant, a composition according to the invention comprises a continuous fatty phase and a dispersed aqueous phase. Thereby, a composition according to the invention is in the form of an oily (or fatty) phase which additionally comprises at least one dispersed aqueous phase, e.g. in encapsulated form such as e.g. in the form of matrix spheres (or beads or small balls) or such as cores/shells (or capsules) e.g. in the form of capsules as described in WO2010/063937.

Thereby, a solid composition according to the invention may also comprise at least one aqueous phase dispersed in the solid composition, preferably in encapsulated form.

According to one embodiment, the mass percentage of water of the aqueous phase may then be comprised between 0.5% and 50%, preferably between 1%, and 40%, more particularly between 2.5% and 30%, and better between 5% and 20%, by weight relative to the total weight of the composition.

The solid character of a composition according to the invention may be based on a phase consisting predominantly, even totally, of a fatty phase comprising at least one lipophilic gelling agent (also referred to hereinafter as “solid fatty phase”).

Preferably, the solid character of a composition according to the invention is based on the presence of a fatty phase comprising at least one lipophilic gelling agent.

### FATTY PHASE

The dispersed fatty phase of a composition according to the invention has a melting point comprised between 50° C. and 100° C., preferably between 60° C. and 90° C.

The melting point of a fatty phase can be measured using a differential scanning calorimeter (DSC), e.g. the calorimeter marketed under the name “DSC Q2000” by TA instruments. The protocols for sample preparation and for measurement are as follows: a 5 mg sample of the sample to be tested, previously heated to 80° C. and sampled under magnetic stirring using a spatula which is also heated, is placed in a hermetic aluminum capsule, or crucible. In order to ensure the reproducibility of the results, two tests are carried out. The measurements are carried out on the calorimeter mentioned hereinabove. The furnace is subjected to a nitrogen flushing. Cooling is provided by the RCS 90 heat exchanger. The sample is then subjected to the following protocol, being first heated to 20° C. and then subjected to a first temperature rise from 20° C. to 130° C. at a heating rate of 5° C./minute, then cooled from 130° C. to −80° C. at a cooling rate of 5° C./minute and finally subjected to a second temperature rise ranging from −80° C. to 130° C. at a heating rate of 5° C./minute. During the second temperature rise, the variation in the difference in power absorbed by the empty crucible and by the crucible containing the sample is measured as a function of the temperature. The melting point of the compound is the value of the temperature corresponding to the apex of the peak of the curve representing the variation of the difference in absorbed power as a function of the temperature. The end-of-melting temperature corresponds to the temperature at which 95% of the sample has melted.

### LIPOPHILIC GELLING AGENT

A composition according to the invention advantageously comprises at least one lipophilic gelling agent.

The lipophilic gelling agent according to the invention may be chosen from organic or inorganic, polymeric or molecular lipophilic gelling agents; fats which are solid at ambient temperature and pressure; and mixtures thereof.

The fatty substances which are solid at ambient temperature and pressure are preferably chosen from at least one wax, at least one pasty fatty substance, at least one butter, and mixtures thereof.

Advantageously, a lipophilic gelling agent is a heat-sensitive gelling agent, namely one which reacts to heat, and in particular is a gelling agent which is solid at ambient temperature and liquid at a temperature greater than 50° C., preferably greater than 60° C., and better greater than 70° C. Preferably, a heat-sensitive lipophilic gelling agent according to the invention has a melting point comprised between 50° C. and 130° C., and preferably between 60° C. and 120° C.

Lipophilic gelling agent(s) include e.g. of same described in WO2021/234135.

According to a particularly preferred embodiment, the lipophilic gelling agent is chosen from Castor Oil/IPDI copolymer (and) Caprylic/Capric Triglyceride, marketed in particular under the name Estogel M by PolymerExpert, Hydrogenated Castor Oil/Sebacic Acid Copolymer and mixtures thereof, in particular marketed under the names Estogel Green (or Estogel G) and Estogel Green 40, respectively, by PolymerExpert, Caprylic/Capric Triglyceride (and) Polyurethane-79, marketed in particular under the name OILKEMIA™ 5S polymer by the company Lubrizol, Trihydroxystearin, in particular marketed under the name THIXCIN® R by the company Elementis Specialties, and mixtures thereof, and, better, Castor Oil/IPDI Copolymer (and) Caprylic/Capric Triglyceride.

More particularly, a composition according to the invention may comprise from 10% to 90%, preferably from 20% to 80%, more particularly from 30% to 70%, and better from 40% to 60%, by weight of lipophilic gelling agent(s) relative to the total weight of the composition.

### OILS

A composition according to the invention can comprise at least one oil. “Oil” refers to a fat that is liquid at ambient temperature and atmospheric pressure.

As oils according to the invention, mention may be made e.g. of:
hydrocarbon oils of vegetable origin, as described hereinafter;
hydrocarbon oils of animal origin, such as perhydrosqualene and squalane;
synthetic esters and ethers, in particular of fatty acids, such as oils with the formulae R1COOR2 and R1OR2 wherein R1 represents the residue of a C₈ to C₂₉ fatty acid and R2 represents a C₃ to C₃₀ hydrocarbon chain, branched or unbranched, such as Purcellin oil, isononyl isononanoate, isodecyl neopentanoate, isostearyl neopentanoate, isopropyl myristate, ethyl-2-hexyl palmitate, octyl-2-dodecyl stearate, octyl-2-dodecyl erucate, isostearyl isostearate; hydroxy esters such as isostearyl lactate, octylhydroxystearate, octyldodecyl hydroxystearate, diisostearyl malate, triisocetyl citrate, heptanoates, octanoates, decanoates of fatty alcohols; polyol esters such as propylene glycol dioctanoate, neopentyl glycol diheptanoate and diethylene glycol diisonanoate; and pentaerythritol esters such as pentaerythrityl tetrabehenate (DUB PTB) or pentaerythrityl tetraisostearate (Prisorine 3631);
linear or branched hydrocarbons of mineral or synthetic origin, such as paraffin oils, either volatile or non-volatile, and the derivatives thereof, petroleum jelly, polydecenes, hydrogenated polyisobutene such as Parleam oil;
silicone oils, such as volatile or non-volatile polymethylsiloxanes (PDMS) with a linear or cyclic silicone chain, liquid or pasty at room temperature, in particular cyclopolydimethylsiloxanes (cyclomethicones) such as cyclohexasiloxane and cyclopentasiloxane; polydimethylsiloxanes (or dimethicones) containing alkyl, alkoxy or phenyl groups, during or at the end of the silicone chain, groups containing from 2 to 24 carbon atoms; phenylsilicones such as phenyltrimethicones, phenyldimethicones, phenyltrimethylsiloxydiphenylsiloxanes, diphenyldimethicones and diphenylmethyldiphenyltrisiloxanes, 2-phenylethyltrimethyl siloxysilicates, and polymethylphenylsiloxanes;
fatty alcohols with 8 to 26 carbon atoms, such as cetyl alcohol, stearyl alcohol and the mixture (cetyl stearyl alcohol) thereof, or else octyldodecanol;
partially hydrocarbon-based and/or silicone-based fluorinated oils such as same described in document JP-A-2-295912;
and mixtures thereof.

As oils, mention may be made e.g. of same described in WO2021/234135.

A composition according to the invention preferably comprises at least one vegetable oil, such as e.g. copra oil, coconut oil, olive oil, sweet almond oil, castor oil, Babassu oil, and mixtures thereof.

More particularly, a composition according to the invention may comprise from 10% to 90%, preferably from 20% to 80%, more particularly from 30% to 70%, and better from 40% to 60%, by weight of oil(s) relative to the total weight of the composition.

A composition according to the invention may comprise at least one lipophilic gelling agent, preferably heat-sensitive, at least one oil and mixtures thereof.

### BIOLOGICAL/COSMETIC ACTIVE AGENT

A solid composition according to the invention, in particular the fatty phase and/or the dispersed aqueous phase (when present), comprises at least one biological/cosmetic active agent.

As biological/cosmetic active agent according to the invention, mention may be made of moisturizing agents, vitamins, cicatrizing agents, anti-wrinkle or anti-aging agents, depigmenting agents, desquamating agents, antioxidants, active agents stimulating the synthesis of dermal and/or epidermal macromolecules, skin relaxant agents, antiperspirant agents, soothing agents and/or anti-aging agents, and mixtures thereof.

### Anti-Wrinkle or Anti-Aging Agents

As representative of anti-wrinkle or anti-aging agents which can be used in the present invention, mention may be made more particularly of adenosine, retinol and derivatives thereof, ascorbic acid and derivatives thereof, such as magnesium ascorbyl phosphate and ascorbyl glucoside; tocopherol and derivatives thereof, such as tocopheryl acetate; nicotinic acid and the precursors thereof, such as nicotinamide; ubiquinone; glutathione and the precursors thereof such as L-2-oxothiazolidine-4-carboxylic acid; C-glycoside and derivatives thereof, such as described in particular hereinafter; extracts of plants and in particular extracts of Crithmum and olive leaf, and plant-based proteins and the hydrolysates thereof, such as rice or soya protein hydrolysates; or else extracts of seeds of Vigna aconitifolia as same marketed by Cognis under the reference Vitoptine LS9529 and Vit-A-Like LS9737; extracts from algae and more particularly of Laminaria; bacterial extracts; sapogenins, such as diosgenin and extracts of Dioscorea, more particularly of Wild Yam, which contain same; α-hydroxyacids; β-hydroxyacids, such as salicylic acid and n-octanoyl-5-salicylic acid; oligopeptides and pseudodipeptides and the acylated derivatives thereof, more particularly {2-[acetyl-(3-trifluoromethyl-phenyl)-amino]-3-methyl-butyrylamino} acetic acid and lipopeptides marketed by SEDERMA under the commercial names Biopeptide CL, Matrixyl 500 and Matrixyl 3000; lycopene; manganese and magnesium salts, more particularly gluconates; an extract of rye seeds under the commercial name Coheliss from Silab; and extract of leaves of Centella asiatica; oily or aqueous extracts of fruits of Vanilla planifolia, extract of flower of Vanilla planifolia; and mixtures thereof.

### Humectants or Moisturizers

Humectants or moisturizers include in particular glycerin; diglycerin; glycols, such as sorbitol; betaines; urea and derivatives thereof, in particular Hydrovance® marketed by National Starch; monosaccharides such as mannose, AHAs, BHAs, beta-glucan and more particularly sodium carboxymethyl beta-glucan from Mibelle-AG-Biochemistry; polyoxybutylene, polyoxyethylene, or polyoxypropylene glycerol such as WILBRIDE S-753L® from NOF corporation; musk rose oil marketed by Nestlé; marine collagen and chondroitin sulfate (Ateocollagen) beads marketed by Engelhard Lyon under the name marine filler beads; niacinamide; glyceryl-polymethacrylate from Sederma sold under the commercial name Lubragel@ MS; trimethylglycin sold under the commercial name Aminocoat® by Ashahi Kasei Chemicals; extracts of nacre containing a conchiolin sold in particular by Maruzen (Japan) under the commercial name Pearl Extract®; plant extracts such as an extract of Castanea sativa or oily or aqueous extracts of Camellia japonica and more particularly of the Alba plena variety; proteins of hydrolysed hazelnuts; polysaccharides of Polyanthes tuberosa; Argania spinosa kernel oil; homo- and co-polymers of 2-methacryloyloxyethyl phosphorylcholine acid, such as Lipidure HM and Lipidure PBM from NOF; saccharides such as glucose, fructose, mannose or trehalose; glycosaminoglycanes and derivatives thereof such as hyaluronic acid, sodium hyaluronate and acetylated hyaluronic acid, more particularly acid hyaluronic beads as same marketed by Engelhard Lyon; panthenol; allantoin; aloe vera; free amino acids and derivatives thereof; glucosamine; citric acid; ceramides; and mixtures thereof.

### Desquamating Agents

As desquamating agents, mention may be made of beta-hydroxy acids, more particularly salicylic acid and derivatives thereof other than n-octanoyl 5 salicylic acid; urea; glycolic, citric, lactic, tartaric, malic or mandelic acids; 4 (2 hydroxyethyl)piperazine 1 propanesulphonic acid (HEPES); extract of Saphora japonica; honey; N-acetyl glucosamine; sodium methyl glycine diacetate, alpha-hydroxy acids (AHA), Beta-hydroxy acids (BHA), and mixtures thereof.

### Antioxidants

As antioxidants, mention may be made more particularly of tocopherol and esters thereof, more particularly tocopherol acetate; EDTA, ascorbic acid and derivatives thereof, more particularly ascorbyl magnesium phosphate and ascorbyl glucoside; chelating agents, such as BHT, BHA, N,N′ bis(3,4,5-trimethoxybenzyl) ethylenediamine and salts thereof, and mixtures thereof.

### Skin Decontracting or Skin Relaxant Agents

As skin decontracting or skin relaxant agents, mention may be made most particularly of manganese gluconate, wild yam, Crithmum, glycine, alverine, and mixtures thereof.

### Active Agents Which Stimulate the Synthesis of Dermal and/or Epidermal Macromolecules and/or Prevent the Degradation Thereof

As active agents stimulating the synthesis of dermal and/or epidermal macromolecules and/or preventing the degradation thereof, mention may be made of peptides extracted from plants, such as the soya hydrolyzate sold by BASF Beauty Care Solutions under the commercial name Phytokine®, malt extract such as marketed under the name Collalift® by company BASF BCS; rice peptides such Nutripeptide® from SILAB, or else an extract of rice peptides such as Colhibin® from Pentapharm DSM, methylsilanol mannuronate such as Algisium C® marketed by Exsymol; an extract of Vaccinium myrtillus such as same described in the application FR-A-2 814 950; lupin extract marketed by SILAB under the commercial name Structurine®, verbena hydrolate, and mixtures thereof.

### Soothing Agents

As soothing agent, mention may be made of dipotassium glycyrrhizate or acetyl-1-dipeptide-ketyl ester under the name Calmosensine from SEDERMA.

### Depigmenting Agents

Mention may be made, as depigmenting agents, of ceramides, vitamin C and derivatives thereof, in particular Vit CG, CP and 3-O ethyl vitamin C, alpha and beta arbutin, ferulic acid, kojic acid, resorcinol and derivatives thereof, more particularly 4-butyl resorcinol, tranexamic acid and derivatives thereof, calcium D pantheteine sulfonate, lipoic acid, ellagic acid, vitamin B3, phenylethyl resorcinol such as Symwhite 377® from Symrise, a kiwi (Actinidia chinensis) fruit water marketed by Gattefosse, an extract of Paeonia suffructicosa root such as same marketed by Ichimaru Pharcos under the name Botanpi Liquid B®, an extract of licorice (Glycyrrhiza glabra), an extract of brown sugar (Saccharum officinarum), such as an extract of molasses marketed by Taiyo Kagaku under the name of Molasses Liquid, a mixture of undecylenic acid and phenylalanine undecylenoyl, such as Sepiwhite MSH® from Seppic.

Mention may also be made of vitamins B3, B5, B6, B8, C, E, or PP, niacin, carotenoids, polyphenols and minerals such as zinc, calcium, magnesium, etc.

More particularly, it is possible to use an antioxidant complex comprising vitamins C and E, and at least one carotenoid, in particular a carotenoid chosen from β-carotene, lycopene, astaxanthin, zeaxanthin and lutein, flavonoids such as catechins, hesperidin, proanthocyanidins and anthocyanins.

Same may also be at least one prebiotic or a mixture of prebiotics. More particularly, the prebiotics may be chosen from oligosaccharides, produced from glucose, galactose, xylose, maltose, sucrose, lactose, starch, xylan, hemicellulose, inulin, gums e.g. such as acacia gums, or a mixture thereof. More particularly, the oligosaccharide comprises at least one fructooligosaccharide. More particularly, this prebiotic may comprise a mixture of fructooligosaccharide and inulin.

More particularly, a solid composition according to the invention comprises between 0.0001% and 60%, preferably between 0.001% and 50%, more particularly between 0.01% and 40%, more particularly better between 0.1% and 35%, and preferably between 1% and 25%, by weight of biological/cosmetic active agent(s) relative to the total weight of said composition.

### ADDITIONAL COMPOUNDS

A composition according to the invention, more particularly the fatty phase, even the dispersed aqueous phase when present, may further comprise at least one additional compound different from the aforementioned lipophilic gelling agent, oils and biological/cosmetic active agents.

A composition according to the invention may also comprise powders; flakes; coloring agents, in particular chosen from water-soluble or water-insoluble, liposoluble or non-liposoluble, organic or inorganic coloring agents, materials with an optical effect, liquid crystals, and mixtures thereof; particulate agents insoluble in the fatty phase; scenting agents; preservatives; humectants; fillers with a blurring effect; UV filters; effervescent agents; disintegrating agents (e.g. croscarmellose, carboxymethylcellulose, etc.); thinners; disintegration-promoting agents; stabilizers; chelating agents; softeners; modifying agents chosen amongst texture agents (or gelling agents) and more particularly hydrophilic gelling agents, agents modifying the pH, the osmotic force and/or modifiers of the refractive index etc. . . . or any usual cosmetic additive; and mixtures thereof.

“Particulate agents insoluble in the fatty phase”, as defined by the invention, refers to the group consisting of pigments, ceramics, polymers, in particular acrylic polymers, and mixtures thereof.

As hydrophilic gelling agents, mention may be made e.g. of same described in FR3041251.

A composition according to the invention may also comprise at least one solvent apt to evaporate, completely or partially, during the manufacturing process, as described hereinafter. As solvent, mention may be made e.g., of an apolar or slightly polar organic solvent, e.g. cyclohexane, THF (tetrahydrofuran), and mixtures thereof.

Of course, a person skilled in the art would make sure to choose the raw materials, ingredients, active agent(s) and/or the additional compound(s) described hereinabove and/or the amount thereof so that the advantageous properties of the composition according to the invention, are not altered or not substantially altered by the envisaged admixture. Also, a person skilled in the art would make sure to choose the nature and/or the amount of raw materials, active agent(s) and/or additional compound(s) described hereinabove depending on the aqueous or fatty nature of the phase considered and/or with regard to the method of manufacturing the composition. Such adjustments fall within the general knowledge of a person skilled in the art.

The solid character of a composition according to the invention advantageously makes it possible to dispense with conventional packaging. Indeed, the packaging of a solid composition according to the invention may e.g. be limited to a simple packaging such as a sheet of paper.

Advantageously, a solid composition according to the invention does not consist of a fiber lattice.

Preferably, a composition according to the invention does not comprise any surfactant.

Preferably, a solid composition according to the invention does not comprise polyvinyl alcohol (PVA or PVOH), polyacrylate, copolymer of acrylic acid and methacrylic acid, polyvinylidene fluoride (PVDF), polymethyl methacrylate (PMMA), polyvinylpyrrolidone, polyalkylene oxide, polyacrylonitrile (PES or PAN), polymethacrylate of polyethylene terephthalate, polyethylene, polypropylene, nylon, hyaluronic acid, starch and derivatives thereof, pullulan, gelatin, cellulose (e.g. rayon), hydroxypropyl methylcellulose, methylcellulose, carboxymethyl cellulose, cotton, and one of the mixtures thereof.

A composition according to the invention does not comprise (or does not rest on) a water-soluble solid support, e.g. such as a hydrogel, more particularly based on starch, where appropriate destructured, and/or poloxamer 407.

### MANUFACTURING METHOD

A composition according to the invention is obtained by any method known to a person skilled in the art, suitable for the manufacture of a solid composition, provided the adaptations needed to ensure the formation of the cavities and thus to satisfy the required criterion of porosity.

Preferably, the method of manufacturing a solid composition according to the invention does not comprise a step of aeration/injection of a gas, e.g. air, where appropriate followed by a step of forming and then drying.

### Method No. 1

According to a first variant, a method of manufacturing a composition according to the invention comprises at least the steps consisting in:
(i) having available a composition in liquid or semi-liquid form;
(ii) casting the composition in a mold and drying until a solid composition is obtained,
(iii) cutting the solid composition into the desired shape, and
(iv) forming cavities to obtain a solid composition according to the invention.

Step (i) is advantageously carried out under hot conditions.

Advantageously, steps (iii) and (iv) are simultaneous.

Advantageously, steps (ii), (iii) and (iv) are simultaneous, in which case the mold has a shape suitable for ensuring the formation of the cavity(ies).

Step (iv) is advantageously carried out by means of a clay cutter.

Method No. 1 is advantageous in that the waste obtained at the end of step (iv) can be recovered during a subsequent manufacture. Indeed, the off-cuts can be heated again and added again in a subsequent method No. 1 at step (i) and/or (ii).

### Method No. 2

According to a second variant, a method of manufacturing a composition according to the invention is an extrusion method.

Such a manufacturing method according to the invention comprises at least the steps consisting of:
(a) having a composition in liquid or semi-liquid form;
(b) passing the composition of step (a) through an extrusion device equipped with a die (or extrusion grating) with a shape suitable for forming a solid bar, and
(c) cutting the bar obtained in step (b) to obtain a solid composition according to the invention.

For obvious reasons, step (b) is carried out at a temperature suitable for obtaining a composition with sufficient viscosity and apt to keep the shape given by the die.

For obvious reasons, the shape of the die of the extrusion device is also suitable for the formation of the cavities.

Advantageously, step (b) is carried out at a temperature lower than the ambient temperature, and e.g. at a temperature lower than or equal to 15° C., or even lower than or equal to 10° C.

In step (b), the extrusion device comprises a die having a shape suitable for forming at the solid bar the cavity(ies) and the desired level of porosity.

As an illustration, an extrusion method according to the invention comprises a die such as the die illustrated in FIG. 4.

Step (c) can be carried out using a knife or a wire, such as e.g. a butter cutting wire.

Preferably, steps (b) and (c) are simultaneous.

Preferably, the method of manufacturing a composition according to the invention is the extrusion method No. 2 described hereinabove.

According to a particular embodiment, applicable to methods No. 1 and No. 2 described above, step (i)/(a) consists in having a fluid composition which may comprise at least one solvent apt to evaporate, completely or partially, during step (ii)/(b), or even in addition during step (iii) and/or (iv)/(c).

As solvent, mention may be made e.g., of an apolar or slightly polar organic solvent, e.g. cyclohexane, THF (tetrahydrofuran), and a mixture thereof.

In the above methods No. 1 and No. 2, when the composition comprises at least one fatty phase comprising at least one lipophilic gelling agent, step (i)/(a) may require heating said composition to a temperature higher than the highest melting point of the lipophilic gelling agents present in said composition.

According to a particular embodiment, the extrusion method No. 2 can be carried out by a 3D printing technique.

### USE

A composition according to the invention is intended for care of a keratin material, more particularly of the skin and/or lips.

More particularly, depending on whether the solid composition according to the invention is intended for direct application to a keratin material or requires a prior mixing with an aqueous medium, a composition according to the invention may e.g. be a cream, a lotion, a serum and a gel for the skin (hands, face, feet, etc.), a hair color product (hair dyes and bleaching agents), a hair care product (lotions, creams, oils), a styling product (lotions, hairsprays, brillantines), a shaving product (soaps, foams, lotions, etc.), a product intended to be applied to the lips, a product for whitening the skin, an anti-wrinkle product. More particularly, the cosmetic compositions of the invention may be an anti-aging serum, a youth serum, or a moisturizing serum.

Advantageously, a solid composition according to the invention is not in the form of a mask.

A solid composition according to the invention is not a composition intended for only the make-up application to a keratin material. On the other hand, a solid composition according to the invention can be a BB cream or a CC cream and thus further comprise at least one pigment.

According to a first embodiment, more particularly when a composition according to the invention is of the single-dose type, a solid composition according to the invention is intended for direct contact with a keratin material.

According to a second embodiment, a solid composition according to the invention is intended to be brought into contact with an aqueous phase, more particularly with water, before, simultaneously and/or after the application thereof to the keratin material to be treated. When a composition according to the invention is not of the single-dose type, the bringing in contact with water is carried out during the first use only, and presupposes a packaging of the “solid composition/water” mixture in a suitable container. For obvious reasons, thereof requires a solid composition of suitable formula and a sufficient quantity of water to ensure the transition from a solid composition to a liquid composition, e.g. in gel form. Thereof also requires the presence in the solid composition of at least one preservative in order to guarantee good preservation over time of the liquid composition thereby formed.

Finally, the present invention also relates to a non-therapeutic cosmetic treatment method for care of a keratin material, more particularly of the skin and/or the lips, comprising at least one step of applying to said keratin material of at least one composition as defined hereinabove.

According to a particular embodiment, the present invention relates to a product comprising, or deriving from the bringing into contact between, at least one first solid composition as described hereinabove and at least one second liquid composition.

### First Solid Composition

The first composition, as defined by the present invention, may equally well be referred to as “first solid composition” or “solid composition”, and corresponds to a solid composition as described hereinabove.

### Second Liquid Composition

The second composition, as defined by the present invention, may equally well be referred to as “second liquid composition” or “liquid composition”.

According to a first variant, the second composition is aqueous.

According to a second variant, the second composition is anhydrous.

According to a third variant, the second composition is an emulsion. An emulsion may be a direct (i.e. oil-in-water), inverse (i.e. water-in-oil) or multiple emulsion, more particularly a double emulsion (i.e. water-in-oil-in-water, oil-in-water-in-oil or oil-in-oil-in-water).

According to a particular embodiment, the second composition is a stable emulsion, preferably direct, highly concentrated in dispersed phase, and in particular is an HIPE (i.e. “High Internal Phase Emulsion”) or an apollonian emulsion.

A second composition according to the invention is not in solid form at ambient temperature and at ambient pressure, i.e. same is apt to flow under its own weight thereof.

A second composition according to the invention is in a liquid form, preferably in the form of a gel.

The viscosity of a second composition or of at least the phase thereof when in the form of an emulsion, can vary significantly, which makes it possible to obtain varied textures.

The viscosity is measured at ambient temperature and at ambient pressure according to the method described in WO2017/046305.

For obvious reasons, the viscosity of the second composition is adjusted to make possible an easy withdrawal/administration of the product according to the invention.

According to one embodiment, the second composition advantageously has a viscosity comprised from 5,000 MPa·s to 500,000 MPa·s, preferentially from 10,000 MPa·s to 300,000 MPa·s, better still from 25,000 MPa·s to 100,000 MPa·s, and more particularly from 50000 MPa·s to 75,000 MPa·s, as measured at 25° C., according to the method described hereinabove.

Preferably, the second composition may reduce the solidity of the first composition. More particularly, the second composition may advantageously comprise at least one starting material apt to reduce the solidity of the first composition. Such embodiment is advantageous in that same makes it possible to change the solid texture of the first composition toward a softer texture. One can thereby speak of a “transformative effect”.

A product according to the invention may advantageously comprise a “first composition(s)/second composition(s)” weight ratio comprised between 0.01 and 10, preferably between 0.1 and 5 and better between 0.5 and 1.

According to a particular embodiment, the first composition may comprise high contents of biological/cosmetic active agent(s) as described hereinbelow. Thereby, the first composition can be considered to be a “booster”.

Given the foregoing, a product according to the invention is an extemporaneous product, i.e. when the product is first used, it is the user who will bring the first composition into contact with the second composition.

Thereby, the invention further relates to a kit comprising:
at least one first solid composition as described hereinabove;
at least one second solid composition as described hereinabove; and
optionally, at least one container.

A container may be referred to, without distinction, as “container”, “packaging” or “primary packaging”.

The invention further relates to the use of the kit described hereinabove, for obtaining a product according to the invention.

Preferably, a kit according to the invention can further comprise at least one means of application/administration.

In the case where the product is intended for cosmetic use, a kit according to the invention may also comprise at least one means of application to a keratin material, e.g. chosen from the group formed by spatulas, brushes, feathers or sponges.

According to a particular variant, the kit may comprise a plurality of first compositions, identical or different, and/or a plurality of second compositions, identical or different, and/or at least one means of application.

Thereby, the invention further relates to a kit comprising a plurality of first compositions in the form of single doses comprising different cosmetic/biological active agents and/or different contents of cosmetic/biological active agent(s).

Thereby, such a kit according to the invention advantageously makes it easy for the user to adapt to the desired effect.

In the case of a cosmetic product, such particular embodiment allows the user to decide on the desired cosmetic effect, in particular according to his/her state of mind (mood), his/her style of clothing and/or the time of day.

### BRIEF DESCRIPTION OF FIGURES

FIG. 1 shows a composition according to the invention, with a parallelepiped shape having a cavity of cylindrical shape. A is the side view and B is the top view.

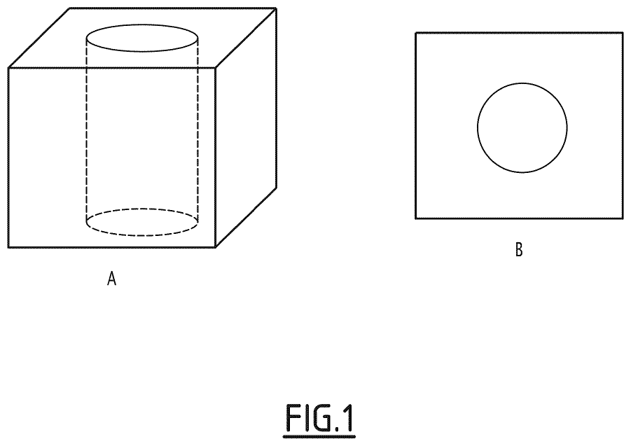

FIG. 2 represents an ovoid-shaped composition according to the invention with cavities (top view), representing the word “SOAP”.

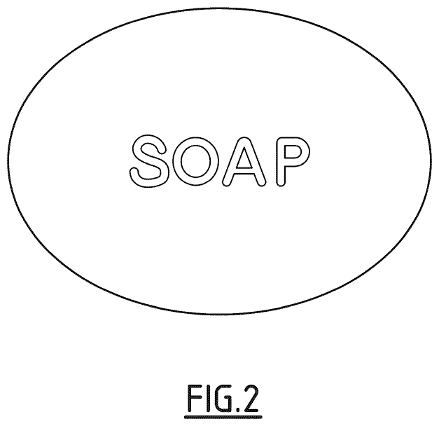

FIG. 3 shows a composition according to the invention with honeycomb cavities (top view).

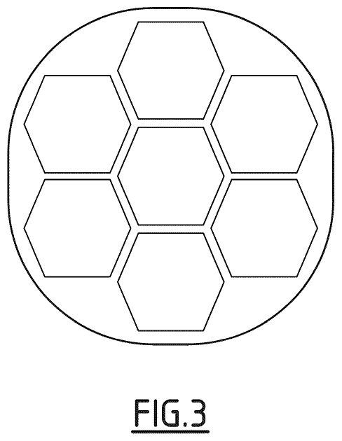

FIG. 4 shows a die used for the method of the invention.

FIG. 5 shows a comparative die.

FIG. 6 shows other examples of solid compositions according to the invention.

### EXAMPLES

### Example 1—Comparative Study

### Step 1—Manufacturing

| NOM | INCI | % | Phase |
|---|---|---|---|
| LABRAFAC CC | Caprylic/CapricTriglyceride | QSP* | A |
| CASTOR OIL | RicinusCommunis(Castor) SeedOil | 8.00 | A |
| ESTOGEL M | Castor Oil/IPDI Copolymer (and) | 30.00 | A |
| | Caprylic/CapricTriglyceride | | |
| ESTOGEL GREEN | HydrogenatedCastor | 8.00 | A |
| | Oil/SebacicAcid | | |
| | Copolymer | | |
| LUSPLAN DD-DA7 | Dimer DilinoleylDimer Dilinoleate | 3.70 | A |
| Coenzyme Q10 | Ubiquinone | 0.2 | B |
| COPHEROL 1250 C | Tocopheryl Acetate | 0.50 | B |
| PARFUM | Fragrance | 0.50 | B |

*Quantum satis

Adding the ingredients of phase A into a container and heat the mixture with stirring at 100° C. until reaching homogenization;

Cooling to 80° C. and adding the ingredients of phase B with stirring until reaching homogenization;

### Step 2—Extrusion

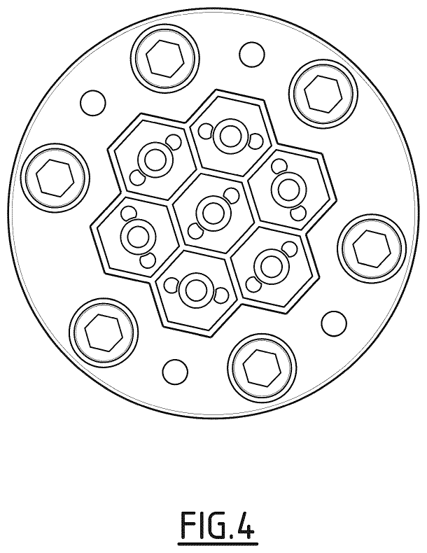

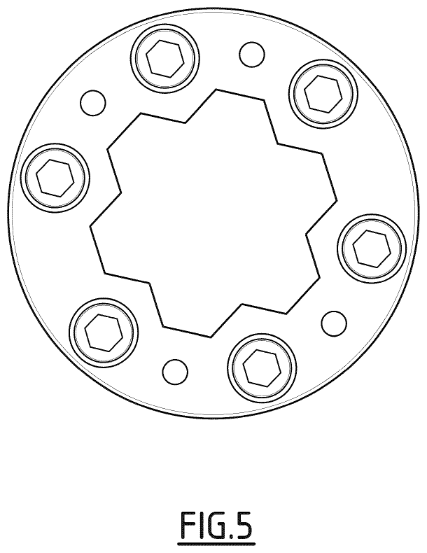

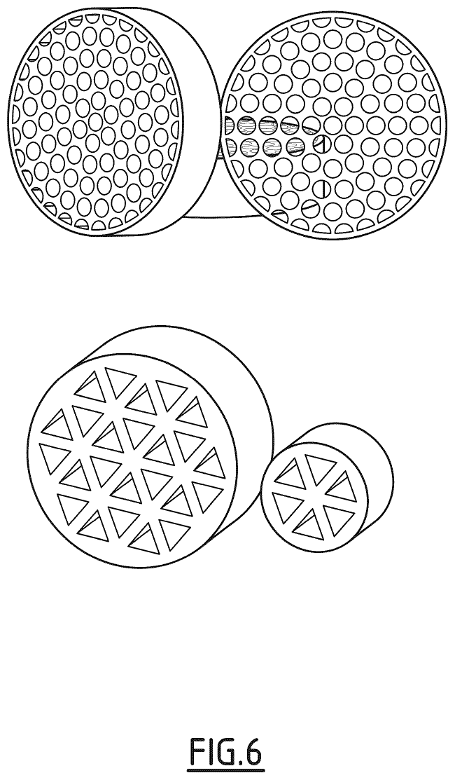

The mixture obtained in step 1 is injected into an extruder provided, in a first case, with a die according to the invention as illustrated in FIG. 4 and, in a second case, with a comparative die as illustrated in FIG. 5;
At the outlet of the die, the respectively extruded bars are cut so as to obtain the weights and volumes described in Table 1 below.
Single-dose solid care compositions are obtained in the form of a solid composition according to the invention with a honeycomb structure (test 1, like e.g. same described in FIG. 6) and a comparative solid composition with a general shape identical to test 1 but with no cavity (soaps 2 and 3), respectively.

| | Test 1 | Test 2 | Test 3 |
|---|---|---|---|
| | (invention) | (comparative) | (comparative) |
| Weight (mg) | 250 | 250 | 1000 |
| Volume (in | 1 | 0.25 | 1 |
| cm³) | | | |
| Porosity (%) | >75 | 0 | 0 |

### Step 3—Application

Step 3 is carried out on a panel of 18 women between the ages of 22 and 47 (hereinafter the “candidates”), 3 groups being then formed, each group being formed of 6 women (groups A, B and C).

For each group, the candidates apply the composition tested on the skin of the face.

Each of the groups A, B and C uses the tests 1, 2 and 3, but each time in different orders.

During the step 3, the candidates assess the performance of tests 1, 2 and 3 in terms of (i) gripping capacity, (ii) homogeneity of the applied deposition, (iii) ease of application and (iii) condition of the composition after use.

### Scoring Criteria

| SCORING | | | |
|---|---|---|---|
| CRITERIA | 0 | 1 | 2 |
| Grip | Satisfactory | Average | Poor |
| Homogeneity | Satisfactory | Average | Poor |
| of the | | | |
| deposition | | | |
| Ease of | Satisfactory | Average | Poor |
| application | | | |
| Condition of | Total | Some residue | General |
| the | disappearance | | structure kept |
| composition | | | |
| after use | | | |

The means of the results obtained with the three groups A, B and C are presented in the following table, bearing in mind that the observed trends are identical between the three groups.

| | Test 1 | Test 2 | Test 3 |
|---|---|---|---|
| | (Invention) | (Comparative) | (Comparative) |
| Grip | 0 | 2 | 0 |
| Homogeneity of the | 0 | 1 | 1 |
| deposition | | | |
| Ease of application | 0 | 1 | 1 |
| Condition after use | 0 | 1 | 2 |

With comparative tests 2 and 3, it is observed that when the size is too small, the grip is poor and handling is difficult and when the product is too bulky, it is not possible to consume same completely during a single-dose use, which generates a lot of losses.

Furthermore, tests 2 and 3 have average properties in terms of deposition homogeneity and ease of application.

Test 1 according to the invention makes it possible to combine the advantages of comparative tests 2 and 3 without the drawbacks thereof.

A composition according to the invention, due to the presence of cavities in the contents considered, makes it possible to obtain solid care compositions combining an ability to generate a homogeneous deposition with less material while generating less loss and having a homogeneity and satisfactory ease of application, without prejudice to the grip-related properties.

A solid composition according to the invention thus consumes less raw materials and is thus both ecologically and economically advantageous.

### Example 2—Product/Kit With Vitamin C Booster

A first solid composition according to the table below, identical in format to that described in Test 1 above, is available.

First solid composition (Vitamin C Booster)

| Raw material | Function | % mass |
|---|---|---|
| GLYCERIN | Plasticizer | 20 |
| PHYTOWAX OLIVE | Fatty substance | 32 |
| LANETTE 16-CETYL | Fatty substance (texture agent), | 27 |
| ALCOHOL | moisturizing and foaming agent | |
| COCOA BUTTER | Fatty substance (texture agent) | 11 |
| Ascorbyl Tetraisopalmitate | Lipophilic vitamin C | 6 |
| (NIKKOL VC-IP) | | |
| VIVASTAR CS 40 DI | Disintegrant swelling | 4 |

| | |
|---|---|
| Total | 100 |

A second liquid composition is available according to the table below.

Second liquid composition

| Raw material | % mass |
|---|---|
| Water | Q.S.* |
| Glycerol | 5 |
| Propanediol | 10 |
| Hyaluronic acid (HMW: 1-2 MDa) | 0.3 |
| (Hasocri, Soliance) | |
| Xanthan | 0.45 |
| Oligogelin (Oligogeline, BiotechMarine) | 5 |
| Ethanol | 2 |
| Total | 100 |

### Protocol for the Preparation of the Second Composition

Xanthan and hyaluronic acid were first dispersed in glycols (glycerol and propanediol) by stirring with a blade stirrer. The oligogelin was then added and stirring continued for about 30 min. Finally, water and ethanol were added and the mixture was mixed with a blade stirrer for about 2 hours.

The second composition thereby obtained is transparent.

### Use

Starting from a kit according to the invention, the first composition is mixed extemporaneously with approximately 500 mg of the second composition, thereby obtaining a product according to the invention.

It is observed that the second composition makes it possible to reduce the solidity of the first composition. More particularly, the second composition makes it possible to make the solid texture of the first composition change toward a soft texture, easily applicable and very fresh and comfortable.

1. A solid cosmetic care composition of a keratin material, comprising at least one biological/cosmetic active agent, and at least one cavity such that said composition comprises a porosity greater than or equal to 20% by volume relative to the total volume of said solid composition.

2. The solid composition according to claim 1, wherein the porosity is greater than or equal to 30% by volume relative to the total volume of said solid composition.

3. The solid composition according to claim 1, wherein the cavity(ies) is/are through.

4. The solid composition according to claim 1, said composition comprising one or a plurality of cavities, either identical or different.

5. The solid composition according to claim 1, wherein the cavity(ies) is/are round, oblong, ovoid, triangular, polygonal, honeycomb, or in the shape of a paving (or cutout).

6. The solid composition according to claim 1, wherein the ratio between the volume of the cavity(ies) and the total volume of the composition is comprised between 0.2 and 0.9.

7. The solid composition according to claim 1, wherein the biological/cosmetic active agent is chosen from moisturizing agents, vitamins, anti-wrinkle or anti-aging agents, cicatrizing agents, depigmenting agents, desquamating agents, antioxidants, active agents stimulating the synthesis of dermal and/or epidermal macromolecules, skin decontracting agents, anti-perspirant agents, soothing agents and/or anti-aging agents, and mixtures thereof.

8. The composition according to claim 1, wherein said composition comprises between 0.0001% and 60% by weight of biological/cosmetic active agent(s) relative to the total weight of said composition.

9. The solid composition according to claim 1, wherein said composition comprises at least one lipophilic gelling agent at least one oil, and a mixture thereof.

10. The solid composition according to claim 1, wherein said composition further comprises at least one aqueous phase dispersed in the solid composition.

11. The solid composition according to claim 1, wherein said composition is a single-dose composition.

12. A kit comprising:
at least a first the solid composition according to claim 1;
at least one second liquid composition; and
optionally, at least one container.

13. A packaging comprising a plurality of the solid compositions according claim 1, said compositions comprising different biological/cosmetic active agent(s) and/or different contents of biological/cosmetic active agent(s).

14. A method of manufacturing the solid cosmetic composition according claim 1, comprising at least the steps consisting of:
(i) having available a composition in liquid or semi-liquid form;
(ii) casting the composition in a mold and drying until a solid composition is obtained,
(iii) cutting the solid composition into the desired shape, and
(iv) forming cavities for obtaining the solid composition.

15. A method of manufacturing the solid cosmetic composition according to claim 1, comprising at least the steps consisting of:
(a) having a composition in liquid or semi-liquid form;
(b) passing the composition of step (a) through an extrusion device equipped with a die with a shape suitable for forming a solid bar, and (c) cutting the bar obtained during step (b) to obtain the solid composition.

16. A non-therapeutic method for cosmetic care treatment of a keratin material, comprising at least one step of applying to said keratin material at least the solid composition according to claim 1.
